(19) United States
(12) Patent Application Publication
(10) Pub. No.: US 2017/0224987 A1
(43) Pub. Date: Aug. 10, 2017

(54) METHOD AND APPARATUS FOR TREATING SLEEP APNEA

(71) Applicant: Invicta Medical, Inc., Portola Valley, CA (US)

(72) Inventors: Steven Thomas Kent, Portola Valley, CA (US); Karena Yadira Puldon, Northridge, CA (US); Harold Byron Kent, Portola Valley, CA (US)

(21) Appl. No.: 15/498,342

(22) Filed: Apr. 26, 2017

Related U.S. Application Data
(63) Continuation-in-part of application No. 14/149,689, filed on Jan. 7, 2014.

(51) Int. Cl. A61N 1/36; A61B 5/0488; A61B 7/00; A61N 1/05

(52) U.S. Cl. CPC A61N 1/3611; A61N 1/0548; A61N 1/36139; A61B 5/0488; A61B 7/003

(57) ABSTRACT

An oral appliance is disclosed that provides electrical stimulation to a patient's tongue in a manner that prevents collapse of the tongue and/or soft palate during sleep. More specifically, the appliance may induce a reversible current or currents in a lateral direction across the tongue in a manner that shortens the patient's Palatoglossus muscle and/or Styloglossus muscle SGM, which in turn elevates the base of the tongue toward the roof of the oral cavity, changes the shape of the tongue, pulls the patient's soft palate downward towards a base of the tongue, and/or decreases a volume of the tongue.

### CROSS-REFERENCE TO RELATED APPLICATION

This application is a continuation-in-part application, and claims the benefit of co-pending and commonly owned U.S. patent application Ser. No. 14/149,689 entitled “METHOD AND APPARATUS FOR TREATING SLEEP APNEA” filed on Jan. 7, 2014, which is hereby incorporated by reference in its entirety.

### TECHNICAL FIELD

The present disclosure relates generally to sleep apnea, and specifically to non-invasive techniques for treating one or more underlying causes and results of sleep apnea.

### BACKGROUND OF RELATED ART

Obstructive sleep apnea (OSA) is a medical condition in which a patient's upper airway is repeatedly partially or fully occluded during sleep. These repeated occlusions of the upper airway may cause sleep fragmentation, which in turn may result in sleep deprivation, daytime tiredness, and malaise. More serious instances of OSA may increase the patient's risk for stroke, cardiac arrhythmias, high blood pressure, and/or other disorders.

OSA may be characterized by the tendency of the soft tissues of the upper airway to collapse during sleep, thereby occluding the upper airway. More specifically, OSA is typically caused by the collapse of the patient's soft palate and/or by the collapse of the patient's tongue (e.g., onto the back of the pharynx), which in turn may obstruct normal breathing.

There are many treatments available for OSA including, for example: surgery, constant positive airway pressure (CPAP) machines, and the electrical stimulation of muscles associated with moving the tongue. Surgical techniques include tracheotomies, procedures to remove portions of a patient's tongue and/or soft palate, and other procedures that seek to prevent collapse of the tongue into the back of the pharynx. These surgical techniques are very invasive. CPAP machines seek to maintain upper airway patency by applying positive air pressure at the patient's nose and mouth. However, these machines are uncomfortable and may have low compliance rates.

Some electrical stimulation techniques seek to prevent collapse of the tongue into the back of the pharynx by causing the tongue to protrude forward (e.g., in an anterior direction) during sleep. For one example, U.S. Pat. No. 4,830,008 to Meer discloses an invasive technique in which electrodes are implanted into a patient at locations on or near nerves that stimulate the Genioglossus muscle to move the tongue forward (e.g., away from the back of the pharynx). For another example, U.S. Pat. No. 7,711,438 to Lattner discloses a non-invasive technique in which electrodes, mounted on an intraoral device, electrically stimulate the Genioglossus muscle to cause the tongue to move forward during respiratory inspiration. In addition, U.S. Pat. No. 8,359,108 to McCreery teaches an intraoral device that applies electrical stimulation to the Hypoglossal nerve to contract the Genioglossus muscle, which as mentioned above may prevent tongue collapse by moving the tongue forward during sleep.

Moving a patient's tongue forward during sleep may cause the patient to wake, which is not desirable. In addition, existing techniques for electrically stimulating the Hypoglossal nerve and/or the Genioglossus muscle may cause discomfort and/or pain, which is not desirable. Further, invasive techniques for electrically stimulating the Hypoglossal nerve and/or the Genioglossus muscle undesirably require surgery and introduce foreign matter into the patient's tissue, which is undesirable.

Thus, there is a need for a non-invasive treatment for OSA that does not disturb or wake-up the patient during use.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

A method and apparatus for reducing the occurrence and/or severity of a breathing disorder, such as OSA, are disclosed herein. In some implementations, a non-invasive and removable intraoral device is disclosed that may provide electrical stimulation to one or more portions of a patient's oral cavity (mouth) in a manner that prevents a collapse of the patient's tongue and/or soft palate during sleep without disturbing (e.g., without waking) the patient. In some aspects, an electric current induced by the device may stimulate the patient's Palatoglossus muscle in a manner that causes the Palatoglossus muscle to stiffen and shorten, which in turn may pull the patient's soft palate and/or palatal arches in a downward direction towards a base of the patient's tongue so as to prevent the soft palate from collapsing and/or from flapping against the back of the patient's throat. Stiffening and/or shortening the Palatoglossus muscle may also cause the patient's tongue to contract and/or stiffen in a manner that prevents collapse of the tongue in a posterior direction (e.g., towards the patient's pharynx).

In other implementations, an electric current induced by the device may stimulate the patient's Styloglossus muscle in a manner that causes the Styloglossus muscle to stiffen and shorten, which in turn may draw the base of the patient's tongue in an upward direction toward the roof of the oral cavity and away from the pharynx so as to prevent the tongue from collapsing and/or from flapping against the back of the patient's throat. Thus, stiffening and/or shortening the Styloglossus muscle may cause the patient's tongue to contract and/or stiffen in a manner that prevents collapse of the tongue in a posterior direction (e.g., toward the patient's pharynx). Electrically stimulating the Styloglossus muscle also can retract the tongue, for example, starting at the tip of the tongue.

In addition, stimulating the Palatoglossus muscle and/or the Styloglossus muscle using the techniques described herein may also raise and/or tense the lateral edges of the tongue, creating a trough and/or lowering a superior surface of the tongue, thereby causing the tongue to cinch downward (e.g., to “hunker down”) in a manner that further prevents obstruction of the patient's upper airway. Stimulation of the patient's Palatoglossus muscle and/or the Styloglossus muscle may also elevate a posterior portion of the patient's tongue, which in turn may further prevent collapse of the tongue onto the back of the patient's pharynx. By preventing collapse of the patient's tongue, patency of the patient's upper airway may be maintained in a non-invasive manner. In some aspects, the device may stimulate the patient's Palatoglossus and/or the Styloglossus muscle without moving the patient's tongue in an anterior direction. In other aspects, the device may stimulate both the Palatoglossus muscle and the Styloglossus muscle simultaneously (or substantially simultaneously). By simultaneously preventing collapse of the patient's soft palate and tongue, patency of the patient's upper airway may be maintained in a non-invasive manner.

### BRIEF DESCRIPTION OF THE DRAWINGS

Various aspects of the present disclosure are illustrated by way of example and are not intended to be limited by the figures of the accompanying drawings, where like reference numerals refer to corresponding parts throughout the drawing figures.

FIG. 1A is a side sectional view depicting a patient's upper airway.

FIG. 1B is a front plan view of the patient's oral cavity.

FIG. 1C is an elevated sectional view of the patient's tongue.

FIG. 1D is a side sectional view of the patient's tongue.

FIG. 2A is a top plan view of a device, situated over a patient's lower teeth, in accordance with aspects of the present disclosure.

FIG. 2B is an elevated perspective view of the device of FIG. 2A.

FIG. 2C is a top plan view of another device, situated over a patient's lower teeth, in accordance with aspects of the present disclosure.

FIG. 2D is an elevated perspective view of the device of FIG. 2C.

FIG. 3A is a side sectional view depicting a patient's upper airway during disturbed breathing.

FIG. 3B is a side sectional view depicting the patient's upper airway in response to electrical stimulation provided in accordance with aspects of the present disclosure.

FIG. 4 is a block diagram of the electrical components of the device of FIGS. 2A-2B.

FIG. 5 is a circuit diagram illustrating an electrical model of the patient's tongue.

FIG. 6A is an illustrative flow chart depicting an example operation in accordance with aspects of the present disclosure.

FIG. 6B is an illustrative flow chart depicting another example operation in accordance with aspects of the present disclosure.

FIG. 7A is an elevated perspective view of another device in accordance with aspects of the present disclosure.

FIG. 7B is an elevated perspective view of the device of FIG. 7A situated over a patient's teeth.

FIG. 7C is a rear plan view of the device of FIG. 7A situated over a patient's teeth.

FIG. 7D is a front plan view of the device of FIG. 7A situated over a patient's teeth.

### DETAILED DESCRIPTION

A non-invasive method and apparatus for treating sleep disorders, such as obstructive sleep apnea (OSA) and/or snoring, are disclosed herein. In the following description, numerous specific details are set forth to provide a thorough understanding of the present disclosure. Also, in the following description and for purposes of explanation, specific nomenclature is set forth to provide a thorough understanding of the present disclosure. However, it will be apparent to one skilled in the art that these specific details may not be required to practice the various aspects of the present disclosure. In other instances, well-known circuits and devices are shown in block diagram form to avoid obscuring the present disclosure. The term “coupled” as used herein means connected directly to or connected through one or more intervening components, circuits, or physiological matter. Any of the signals provided over various buses described herein may be time-multiplexed with other signals and provided over one or more common buses, or may be wirelessly transmitted between a number of component, circuits, sensors, and/or devices in accordance with aspects of the present disclosure. Additionally, the interconnection between circuit elements or software blocks may be shown as buses or as single signal lines. Each of the buses may alternatively be a single signal line, and each of the single signal lines may alternatively be buses, and a single line or bus might represent any one or more of a myriad of physical or logical mechanisms for communication between components. Further, the logic levels and timing assigned to various signals in the description below are arbitrary and/or approximate, and therefore may be modified (e.g., polarity reversed, timing modified, etc.) as desired.

As used herein, the term “substantially lateral direction” refers to a direction across the patient's oral cavity in which the direction's lateral components are larger than the direction's anterior-to-posterior components (e.g., a substantially lateral direction may refer to any direction that is less than approximately 45 degrees from the lateral direction, as defined below with respect to the drawing figures). Further, as used herein, the term “reversible current” means a current that changes or reverses polarity from time to time between two controllable voltage potentials.

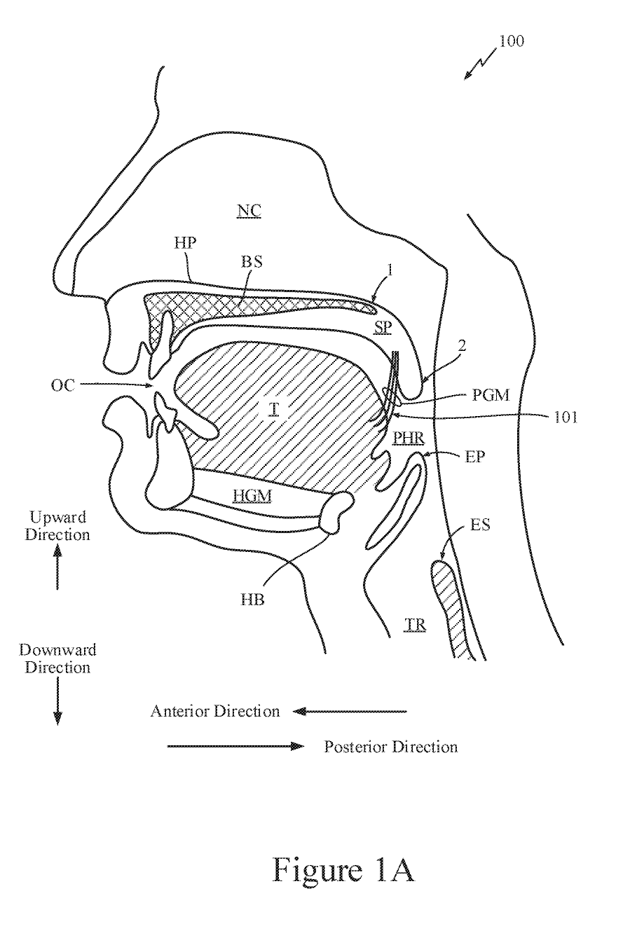

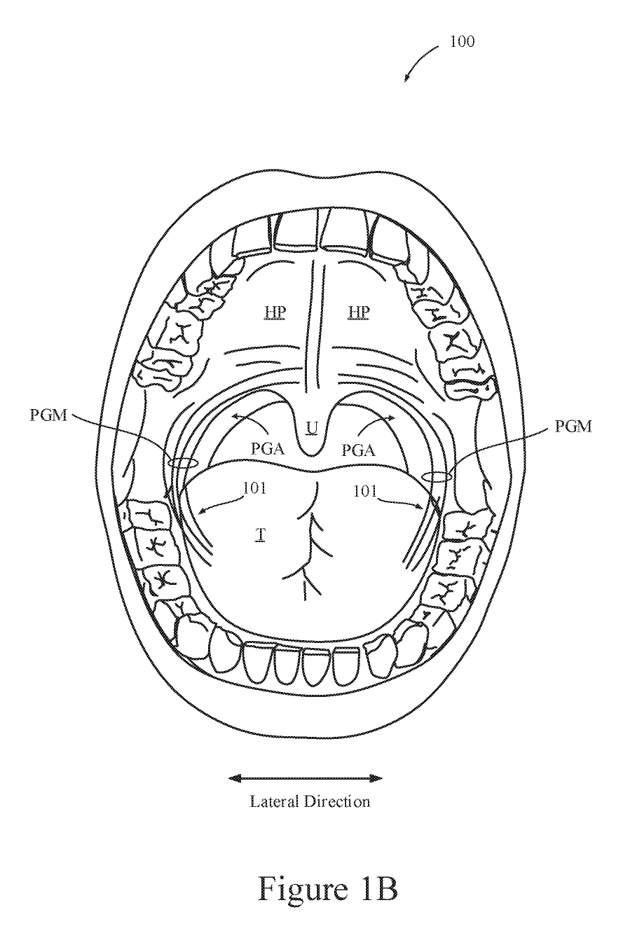

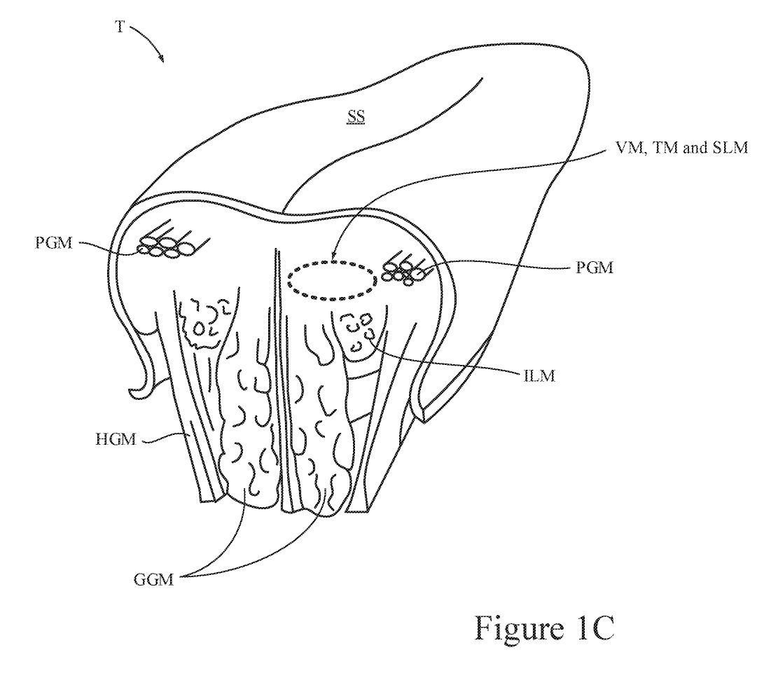

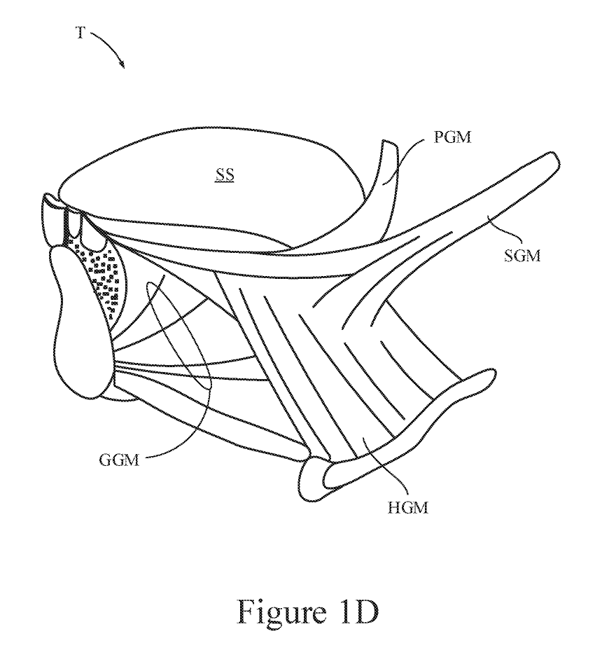

To more fully understand aspects of the present disclosure, the dynamics of OSA are first described with respect to an illustration 100 of a patient's oral cavity shown in FIGS. 1A-1D, which illustrate the anatomical elements of a patient's upper airway 100 (e.g., including the nasal cavity, oral cavity, and pharynx of the patient). Referring first to FIGS. 1A-1B, the hard palate HP overlies the tongue T and forms the roof of the oral cavity OC (e.g., the mouth). The hard palate HP includes bone support BS, and thus does not typically deform during breathing. The soft palate SP, which is made of soft material such as membranes, fibrous material, fatty tissue, and muscle tissue, extends rearward (e.g., in a posterior direction) from the hard palate HP toward the back of the pharynx PHR. More specifically, an anterior end 1 of the soft palate SP is anchored to a posterior end of the hard palate HP, and a posterior end 2 of the soft palate SP is un-attached. Because the soft palate SP does not contain bone or hard cartilage, the soft palate SP is flexible and may collapse onto the back of the pharynx PHR and/or flap back and forth (e.g., especially during sleep).

The pharynx PHR, which passes air from the oral cavity OC and nasal cavity NC into the trachea TR, is the part of the throat situated inferior to (below) the nasal cavity NC, posterior to (behind) the oral cavity OC, and superior to (above) the esophagus ES. The pharynx PHR is separated from the oral cavity OC by the Palatoglossal arch PGA, which runs downward on either side to the base of the tongue T.

Although not shown for simplicity, the pharynx PHR includes the nasopharynx, the oropharynx, and the laryngopharynx. The nasopharynx lies between an upper surface of the soft palate SP and the wall of the throat (i.e., superior to the oral cavity OC). The oropharynx lies behind the oral cavity OC, and extends from the uvula U to the level of the hyoid bone HB. The oropharynx opens anteriorly into the oral cavity OC. The lateral wall of the oropharynx consists of the palatine tonsil, and lies between the Palatoglossal arch PGA and the Palatopharyngeal arch. The anterior wall of the oropharynx consists of the base of the tongue T and the epiglottic vallecula. The superior wall of the oropharynx consists of the inferior surface of the soft palate SP and the uvula U. Because both food and air pass through the pharynx PHR, a flap of connective tissue called the epiglottis EP closes over the glottis (not shown for simplicity) when food is swallowed to prevent aspiration. The laryngopharynx is the part of the throat that connects to the esophagus ES, and lies inferior to the epiglottis EP.

Referring also to FIGS. 1C-1D, the tongue T includes a plurality of muscles that may be classified as either intrinsic muscles or extrinsic muscles. The intrinsic muscles, which lie entirely within the tongue T and are responsible for altering the shape of the tongue T (e.g., for talking and swallowing), include the superior longitudinal muscle SLM, the inferior longitudinal muscle ILM, the vertical muscle VM, and the transverse muscle TM. The superior longitudinal muscle SLM runs along the superior surface SS of the tongue T under the mucous membrane, and may be used to elevate, retract, and deviate the tip of the tongue T. The inferior longitudinal muscle ILM lines the sides of the tongue T, and is attached to the Styloglossus muscle SGM. The vertical muscle VM is located along the midline of the tongue T, and connects the superior and inferior longitudinal muscles together. The transverse muscle TM divides the tongue at the middle, and is attached to the mucous membranes that run along the sides of the tongue T.

The extrinsic muscles, which attach the tongue T to other structures and are responsible for re-positioning (e.g., moving) the tongue, include the Genioglossus muscle GGM, the Hyoglossus muscle HGM, the Styloglossus muscle SGM, and the Palatoglossus muscle PGM. The Genioglossus muscle GGM may be used to protrude the tongue T and to depress the center of the tongue T. The Hyoglossus muscle HGM may be used to depress the tongue T. The Styloglossus muscle SGM may be used to elevate and retract the tongue T. The Palatoglossus muscle PGM may be used to depress the soft palate SP and/or to elevate the back (posterior portion) of the tongue T. Referring also to FIGS. 1A and 1B, the Palatoglossus muscle PGM connects the tongue T to both sides of the Palatoglossus arch PGA, and inserts into lateral posterior regions 101 of the base of the tongue T. The Styloglossus muscle SGM originates at the styloid process of the temporal bone bilaterally, inserts along the lateral aspect of the tongue to the tip (as denoted by regions 101 of the tongue T), and blends with the superior margin of the Hyoglossus muscle and the other intrinsic muscles of the tongue.

It is noted that all of the muscles of the tongue T, except for the Palatoglossus muscle PGM, are innervated by the Hypoglossal nerve (not shown for simplicity); the Palatoglossus muscle PGM is innervated by the pharyngeal branch of the Vagus nerve (not shown for simplicity).

During awake periods, the muscles of the upper airway (as well as the hypoglossal nerve) are active and stimulated, and may maintain upper airway patency by preventing the soft palate SP from collapsing and/or by preventing the tongue T from prolapsing onto the back of the pharynx PHR. However, during sleep periods, a relative relaxed state of the soft palate SP may allow the soft palate SP to collapse and obstruct normal breathing, while a relative relaxed state of the tongue T may allow the tongue T to move in a posterior direction (e.g., onto the back of the pharynx PHR) and obstruct normal breathing.

Accordingly, conventional electrostimulation treatments for OSA typically involve causing the tongue T to move forward in the anterior direction during apnea episodes so that the tongue T does not collapse in the posterior direction. More specifically, some conventional techniques (e.g., disclosed in U.S. Pat. Nos. 5,190,053 and 6,212,435) electrically stimulate the Genioglossus muscle to move the tongue forward in an anterior direction during apnea episodes, while other conventional techniques (e.g., disclosed in U.S. Pat. No. 8,359,108) electrically stimulate the Hypoglossal nerve, which in turn causes the tongue to move forward in the anterior direction by innervating the Genioglossus muscle.

Unfortunately, repeatedly moving the tongue T forward (e.g., in the anterior direction) to prevent its prolapse into the back of the pharynx PHR may undesirably wake-up the patient, which defeats the very purpose of OSA treatments and may also abrade the tongue on the teeth. Indeed, electrically stimulating the relatively large Genioglossus muscle may cause discomfort or pain. In addition, because the Genioglossus muscle GGM is primarily responsible for moving the tongue in an anterior-to-posterior direction, stimulating the Genioglossus muscle GGM (such as by stimulating the Hypoglossal nerve) in an attempt to move the tongue forward during apnea episodes may not only over-stimulate the patient's tongue muscles but may also cause the tongue T to behave erratically (e.g., repeatedly protruding and retracting).

Applicant has discovered that OSA may be more effectively treated by targeting the Palatoglossus muscle PGM for electrical stimulation (e.g., rather than targeting the Genioglossus muscle GGM or the Hypoglossal nerve for electrical stimulation). More specifically, Applicant has discovered that application of one or more voltage differentials across selected portions of the patient's lateral tissues (including, for example, the sublingual tissues) may induce a current across the tongue to electrically stimulate the Palatoglossus muscle PGM in a manner that causes the Palatoglossus muscle PGM to shorten (e.g., to decrease its length). In some aspects, the induced current may flow in a lateral direction across a base portion of the patient's tongue (e.g., proximate to the lateral points at which the Palatoglossus muscle inserts into the tongue T). Shortening the Palatoglossus muscle, using techniques described herein, may (1) stiffen and reduce the volume of the tongue T and (2) may cause the Palatoglossal arch PGA to pull down (e.g., in a downward direction) towards the base of the tongue T.

In some implementations, OSA may also be treated by directly targeting the Styloglossus muscle SGM for electrical stimulation (such as without targeting the Hypoglossal nerve for electrical stimulation). Referring to FIG. 1D, given the more lateral location of the Styloglossus muscle relative to the Genioglossus muscle GGM and the Hypoglossal nerve, the Styloglossus muscle can be electrically stimulated without unintentional stimulation of the Genioglossus muscle GGM or the Hypoglossal nerve. By stimulating the Styloglossus muscle directly, over-stimulation of the patient's tongue may be avoided. As discussed herein, Applicant has discovered that application of one or more voltage differentials across selected portions of the patient's lateral tissues (including, for example, the sublingual tissues) may induce a current across the tongue to electrically stimulate the Styloglossus muscle SGM in a manner that causes the Styloglossus muscle SGM to shorten (e.g., to decrease its length). In some aspects, the induced current may flow in a lateral direction across a base portion of the patient's tongue (e.g., proximate to the lateral points at which the Styloglossus muscle SGM inserts into the tongue T). Shortening the Styloglossus muscle SGM, using techniques described herein, may (1) stiffen, reduce the volume of the tongue T, and/or change shape and (2) may elevate the base of the tongue T up (e.g., in an upward direction) toward the roof of the oral cavity, away from the pharynx.

As described in more detail below, reducing the volume of the tongue T using techniques described herein may prevent the tongue T from prolapsing onto the back of the pharynx PHR. Elevating the base of the tongue T using techniques described herein may also prevent the tongue T from collapsing onto the back of the pharynx PHR. In addition, stimulating the Palatoglossus muscle PGM and/or Styloglossus muscle SGM using techniques described herein may also raise and/or tense the lateral edges of the tongue, creating a trough and/or lowering the superior surface SS of the tongue T, thereby causing the tongue to cinch downward (e.g., to “hunker down”) in a manner that further prevents obstruction of the patient's upper airway. Further, stimulating the Palatoglossus muscle PGM using techniques described herein may also pull down the Palatoglossal arch PGA, thereby preventing the soft palate SP from collapsing onto the back of the pharynx PHR.

Perhaps equally important, because at least some aspects disclosed herein do not target either the Hypoglossal nerve or the Genioglossus muscle GGM for electrical stimulation, these aspects may not cause the tongue T to move forward in the anterior direction during application of the electrical stimulation, which in turn may reduce the likelihood of undesirably waking-up the patient. In some aspects, the voltage differential may be applied across the patient's lateral lingual tissues in a manner that maintains the patient's tongue in a substantially stationary position while shortening the patient's Palatoglossus muscle PGM. In this manner, aspects of the present disclosure may maintain a patient's upper airway patency in a subtle yet therapeutic manner. Although electrical stimulation of the Palatoglossus muscle PGM using techniques described herein is not intended to stimulate the Genioglossus muscle GGM, any inadvertent stimulation of the Genioglossus muscle GGM will be relatively small and, at most, may serve to maintain the tongue T in a substantially stationary position.

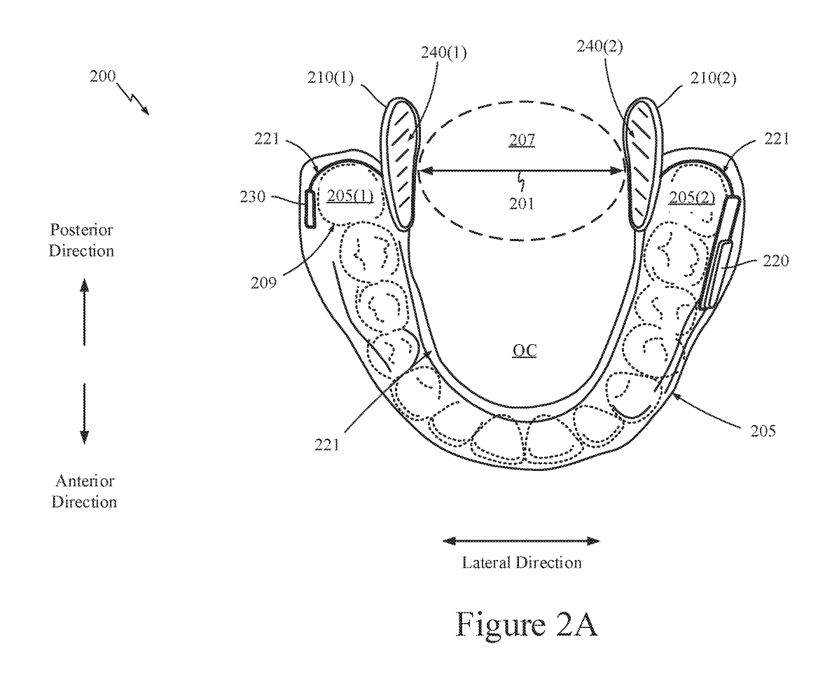

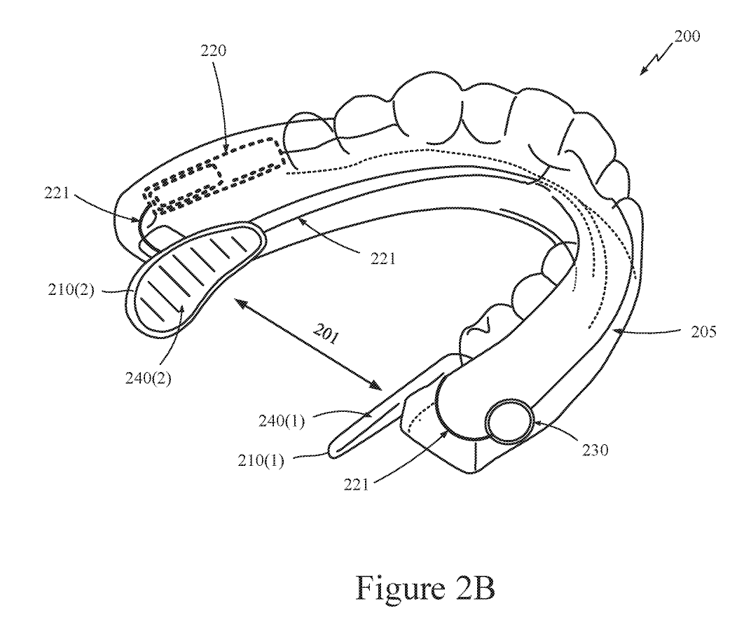

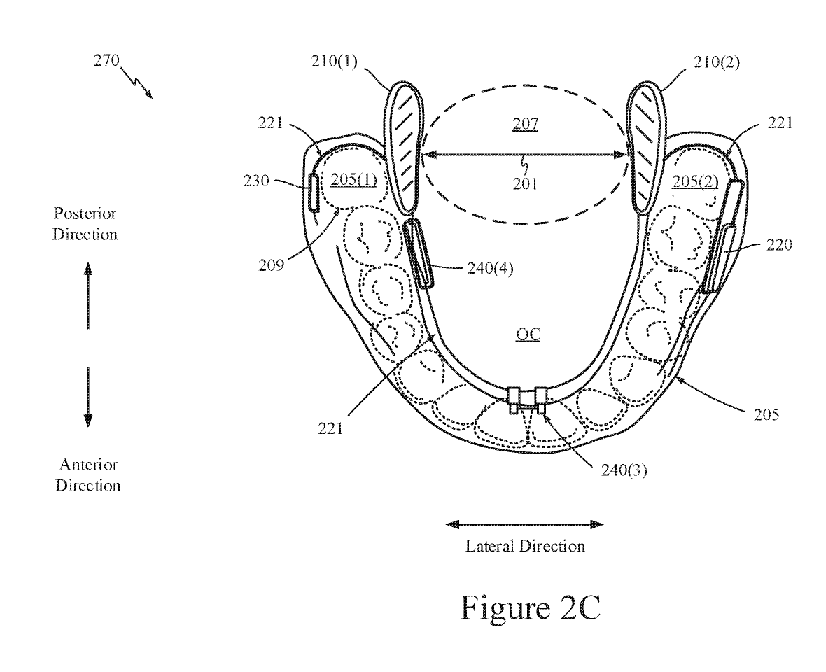

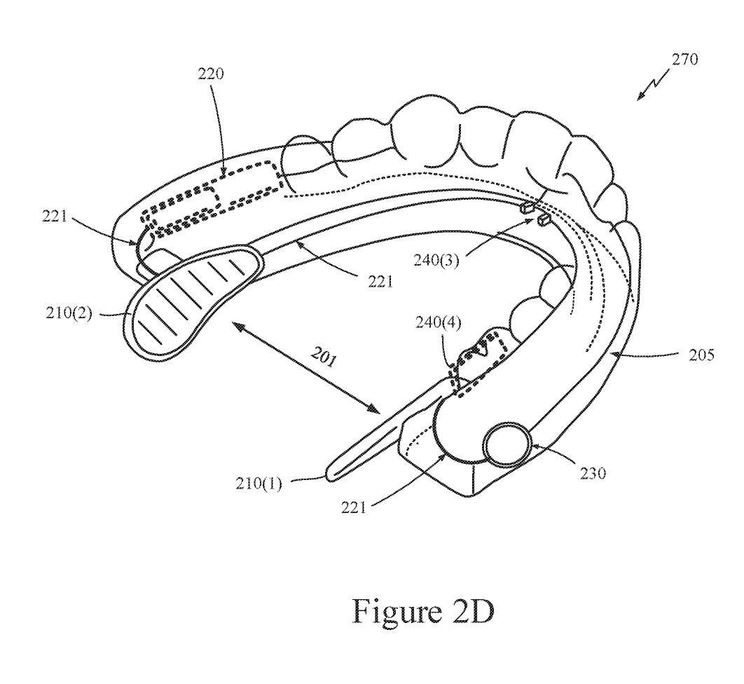

FIGS. 2A-2B show a removable intraoral device 200 in accordance with aspects of the present disclosure. The device 200 may be used to treat OSA by electrically stimulating the Palatoglossus muscle PGM and/or the Styloglossus muscle SGM in a manner that prevents softening of the tongue, prevents a reduction of muscle tone, and prevents collapse of the tongue into the back of the pharynx (or into other posterior portions of the person's upper airway). In some implementations, the device 200 may be configured to target the Palatoglossus muscle for electrical stimulation, for example, by inducing a current directed at the lateral points at which the Palatoglossus muscle inserts into the tongue. Electrical stimulation of the Palatoglossus muscle may maintain a patient's upper airway patency by decreasing the volume of the tongue, by stiffening the tongue, by elevating a posterior portion of tongue, by preventing the soft palate from collapsing onto the back of the pharynx, by pulling the soft palate down towards base of tongue, by causing the tongue to cinch in a downward direction, and/or by raising and tensing the lateral edges of the tongue (thereby creating a trough in the tongue).

In other implementations, the device 200 may be configured to target the Styloglossus muscle for electrical stimulation, for example, by inducing a current directed at the lateral points at which the Styloglossus muscle inserts into the tongue. Electrical stimulation of the Styloglossus muscle SGM may maintain a patient's upper airway patency by decreasing the volume of the tongue, by stiffening the tongue, by elevating a posterior portion of tongue, by drawing the Palatoglossal arches superiorly (such as away from the posterior pharynx), and/or by elevating the base of the tongue toward the roof of the oral cavity (such as away from the pharynx).

In still other implementations, the device 200 may be configured to electrically stimulate the Palatoglossus muscle and the Styloglossus muscle concurrently (or substantially concurrently). In some aspects, the device 200 may be configured to electrically stimulate the Palatoglossus muscle and the Styloglossus muscle at the same times. In other aspects, the device 200 may be configured to electrically stimulate the Palatoglossus muscle and the Styloglossus muscle at different times (such as overlapping times or staggered times). By targeting both the Palatoglossus muscle and the Styloglossus muscle for electrical stimulation, the efficacy of device 200 may be improved, for example, as compared with electrically stimulating one of the Palatoglossus muscle and the Styloglossus muscle. For one example, because the Palatoglossus muscle and the Styloglossus muscle may control a number of similar aspects of the tongue (such as decreasing the volume of the tongue, stiffening the tongue, and elevating a posterior portion of tongue), electrically stimulating both the Palatoglossus muscle and the Styloglossus muscle may increase these common effects. For another example, because the Styloglossus muscle may control a number of different aspects of the tongue than the Palatoglossus muscle (such as retracting the tip of the tongue), electrically stimulating both the Palatoglossus muscle and the Styloglossus muscle may provide additional mechanisms for preventing collapse of the tongue (as compared with only targeting the Palatoglossus muscle for electrical stimulation).

The device 200 is shown in FIGS. 2A-2B as including an appliance 205 upon which a number of electrodes or contacts 210(1)-210(2), a control circuit 220, and a power supply 230 may be mounted (or otherwise attached to) so as to form a unitary or divisible removable device that may fit generally within a patient's oral cavity OC (see also FIGS. 1A-1B).

In some implementations, the device 200 may be adapted or configured to be positioned entirety within the patient's oral cavity, for example, so that none of the components associated with the device 200 protrude from the patient's mouth or body (e.g., none of the device components are external to the patient's body). In some aspects, the device 200 may be fitted over a patient's lower teeth and positioned to fit within a sublingual portion of the patient's oral cavity OC, for example, as depicted in FIG. 2A. In other aspects, the device 200 may be of other suitable configurations or structures, and the contacts 210(1)-210(2) may be provided in other suitable positions. Thus, although not depicted in FIG. 2A, the device 200 may be configured to fit within an upper portion of the patient's oral cavity, for example, by configuring the appliance 205 to fit over the upper teeth of the patient.

In other implementations, one or more components of the device 200 may protrude slightly outside the lips or mouth of the patient. In some aspects, the control circuit 220, power supply 230, and/or other components may be detached from the device 200 and located outside the patient's mouth. For these aspects, the control circuit 220, power supply 230, and/or other components may be electrically coupled to the contacts 210(1)-210(1) using wired connections (e.g., conductive wires).

Although only two contacts 210(1)-210(1) are shown in the example of FIGS. 2A-2B, it is to be understood that the device 200 may include a greater or fewer number of contacts. For example, in other implementations, the device 200 may include four contacts 210 (or another suitable number of contacts 210) arranged in opposing (e.g., “X”) patterns with respect to the patient's upper airway tissues, wherein pairs of the contacts may be selectively enabled and disabled in a manner that alternately induces two or more currents across the patient's upper airway tissues. In some aspects, each of such contacts may be turned on and/or off independently of the other contacts, for example, to determine a pair (or more) of contacts that, at a particular moment for the patient, correlate to optimum electrical stimulation. The determined pair of contacts may be dynamically selected either by (1) directly correlating electrical stimulation and immediate respiratory response or by (2) indirectly using the device 200 “to look for” the lowest impedance contact “pair(s).” The determined contacts may or may not be at the ends of an “X” pattern, and may be opposing one another.

The first contact 210(1) and the second contact 210(2), which may be formed using any suitable material and may be of any suitable size and/or shape, are connected to the control circuit 220 by wires 221. The wires 221 may be any suitable wire, cable, conductor, or other conductive element that facilitates the exchange of signals between control circuit 220 and the contacts 210(1)-210(2). The control circuit 220 and contacts 210(1)-210(2) are electrically coupled to power supply 230 via wires 221. Note that the wires 221 may be positioned either within or on an outside surface of the body 205, and therefore do not protrude into or otherwise contact the patient's tongue or oral tissue. The power supply 230 may be mounted in any of several locations and may be any suitable power supply (e.g., a battery) that provides power to control circuit 220 and/or contacts 210(1)-210(2). Multi-directional gating techniques may be used to control voltages and/or currents within wires 221, for example, so that wires 221 may alternately deliver power to contacts 210(1)-210(2) and exchange electrical signals (e.g., sensor signals) between contacts 210(1)-210(2) and control circuit 220.

For the example of FIGS. 2A-2B, the first contact 210(1) may include or also function as a sensor 240(1), and the second contact 210(2) may include or also function as a sensor 240(2), which could sense respiration or other functions of interest. Thus, in some implementations, one or both of contacts 210(1)-210(2) may also function as sensors such as respiration sensors. In some aspects, the active function of the contacts 210(1)-210(2) may be controlled using multi-directional gating techniques. For example, when the first contact 210(1) is to function as a driven contact, the multi-directional gating technique may connect the first contact 210(1) to the output of a circuit such as a voltage and/or current driver (e.g., included within or associated with control circuit 220), for example, to provide a first voltage potential at the first contact 210(1); conversely, when the first contact 210(1) is to function as the respiration sensor or other sensor 240(1), the multi-directional gating technique may connect sensor 240(1) to the input of a circuit such as an amplifier and/or an ADC (analog to digital) converter (e.g., included within or associated with control circuit 220), for example, to sense a respiratory function of the patient.

Similarly, when the second contact 210(2) is to function as a driven contact, the multi-directional gating technique may connect the second contact 210(2) to the output of a circuit such as a voltage and/or current driver (e.g., included within or associated with control circuit 220), for example, to provide a second voltage potential at the second contact 210(2); conversely, when the second contact 210(2) is to function as the respiration sensor or other sensor 240(2), the multi-directional gating technique may connect sensor 240(2) to the input of a circuit such as an amplifier and/or an ADC (analog to digital) converter (e.g., included within or associated with control circuit 220), for example, to sense a respiratory function of the patient.

The respiration sensors or other sensors 240(1)-240(2), as provided within or otherwise associated with the contacts 210(1)-210(2), may be any suitable sensors that measure any physical, chemical, mechanical, electrical, neurological, and/or other characteristics of the patient which may indicate or identify the presence and/or absence of disturbed breathing. These respiration sensors 240(1)-240(2) may also be used to detect snoring. In some implementations, one or both of contacts 210(1)-210(2) may include electromyogram (EMG) sensor contacts that, for example, detect electrical activity of the muscles and/or nerves within, connected to, or otherwise associated with the oral cavity. In some aspects, one or both of contacts 210(1)-210(2) may include a microphone (or any other sensor to sense acoustic and/or vibration energy) to detect the patient's respiratory behavior. In other aspects, one or both of contacts 210(1)-210(2) may include one or more of the following non-exhaustive list of sensors: accelerometers, piezos, capacitance proximity detectors, capacitive sensing elements, optical systems, EMG sensors, etc.

In some other implementations, the contacts 210(1)-210(2) may not include any sensors. In some aspects, the contacts 210(1)-210(2) may continuously provide electrical stimulation to the patient's Palatoglossus muscle PGM and/or Styloglossus muscle SGM via the lingual tissues. In other aspects, a timer (not shown for simplicity) may be provided on the appliance 205 or within control circuit 220 and configured to selectively enable/disable contacts 210(1)-210(2), for example, based upon a predetermined stimulation schedule. In another closed-loop implementation, the contacts 210(1)-210(2) may be selectively enabled/disabled based upon one or more sources of sensor feedback from the patient.

For the example of FIGS. 2A-2B, the first and second contacts 210(1)-210(2) may be mounted on respective lateral arms 205(1) and 205(2) of the appliance 205 of the device 200 such that when the device 200 is placed within a portion of the patient's oral cavity OC, the first and second contacts 210(1)-210(2) are positioned on opposite sides of the posterior region 207 of the patient's oral cavity OC. In other implementations, the first and second contacts 210(1)-210(2) may be separate from the appliance 205 but connected to respective lateral arms 205(1)-205(2), for example, so as to “float” beneath or on either side of the patient's tongue T, or alternatively oriented so as to be positioned on opposite sides of the superior surface of the tongue T. In some implementations, the first and second contacts 210(1)-210(2) are positioned in the posterior sublingual region 207 of the oral cavity OC such that at least a portion of each of the first and second contacts 210(1)-210(2) is positioned posterior to a molar 209 of the patient (or least a position in the oral cavity OC were a last molar would be). In this manner, the first and second contacts 210(1)-210(2) may be in physical contact with the patient's lingual tissues proximate to the lateral regions (e.g., points) 101 at which the Palatoglossus muscle PGM and the Styloglossus muscle SGM insert into the tongue T (see also FIGS. 1A-1B). Further, as depicted in FIGS. 2A-2B, the first and second contacts 210(1)-210(2) may be angularly oriented with respect to the floor of the mouth such that the first and second contacts 210(1)-210(2) substantially face and/or contact opposite sides of the tongue T proximate to the lateral regions (e.g., points) 101 at which the Palatoglossus muscle PGM and the Styloglossus muscle SGM insert into the tongue T (see also FIGS. 1A-1B). In other implementations, the first and second contacts 210(1)-210(2) may be provided in one or more other positions and/or orientations.

For the example depicted in FIG. 2A, at least a portion of each of the first and second contacts 210(1)-210(2) extends beyond (such as in the posterior direction) the last molar location 209 of the patient's oral cavity. Positioning the contacts 210(1)-210(2) on opposite sides of the patient's tongue posterior to a last molar location of the patient is critical to stimulating a patient's Palatogolossus muscle while avoiding electrical coupling with the patient's Hypoglossal nerve. In some aspects, the contacts 210(1)-210(2) can be positioned on opposite lateral sides of the patient's tongue, and angularly oriented with respect to the floor of the mouth.

The control circuit 220 may provide one or more signals to the first and second contacts 210(1)-210(2) to create a voltage differential across the patient's lingual tissues (e.g., across the base of the tongue) in the lateral direction. For purposes of discussion herein, the first contact 210(1) may provide a first voltage potential V1, and the second contact 210(2) may provide a second voltage potential V2. The voltage differential (e.g., V2−V1) provided between the first and second contacts 210(1)-210(2) may induce a current 201 in a substantially lateral direction across the patient's lingual tissues. In some aspects, the current 201 is induced in a substantially lateral direction across the patient's tongue. The current 201, which for some aspects may be a reversible current (as described in more detail below), electrically stimulates the patient's Palatoglossus muscle PGM and/or Styloglossus muscle SGM in a manner that shortens the Palatoglossus muscle PGM and/or Styloglossus muscle SGM respectively.

When the Palatoglossus muscle PGM is stimulated and/or shortened in response to the current 201 induced by the first and second contacts 210(1)-210(2), the Palatoglossus muscle PGM causes the tongue T to stiffen in a manner that decreases the tongue's volume and/or alters its shape, and that may also slightly cinch a portion of the tongue T closer to the floor of the oral cavity OC. One or more of decreasing the tongue's volume and slightly cinching the tongue T downward towards the floor of the oral cavity OC may prevent the tongue T from prolapsing onto the back of the pharynx PHR, thereby maintaining patency of the patient's upper airway (e.g., without moving the tongue forward in the anterior direction). The shortening of the Palatoglossus muscle PGM may also pull the patient's Palatoglossal arch PGA in a downward direction towards the base of the tongue T, which in turn may prevent the soft palate SP from collapsing and obstructing the patient's upper airway.

When the Styloglossus muscle SGM is stimulated and/or shortened in response to the current 201 induced by the first and second contacts 210(1)-210(2), the Styloglossus muscle SGM causes the tongue T to stiffen in a manner that decreases the tongue's volume and/or alters its shape, and that may also slightly cinch a portion of the tongue T closer to the floor of the oral cavity OC. One or more of decreasing the tongue's volume and slightly cinching the tongue T downward toward the floor of the oral cavity OC may prevent the tongue T from prolap sing onto the back of the pharynx PHR, thereby maintaining patency of the patient's upper airway (e.g., without moving the tongue forward in the anterior direction). The shortening of the Styloglossus muscle SGM may elevate the base of the tongue T up (e.g., in an upward direction) toward the roof of the oral cavity, away from the pharynx which in turn may prevent the tongue from collapsing and obstructing the patient's upper airway.

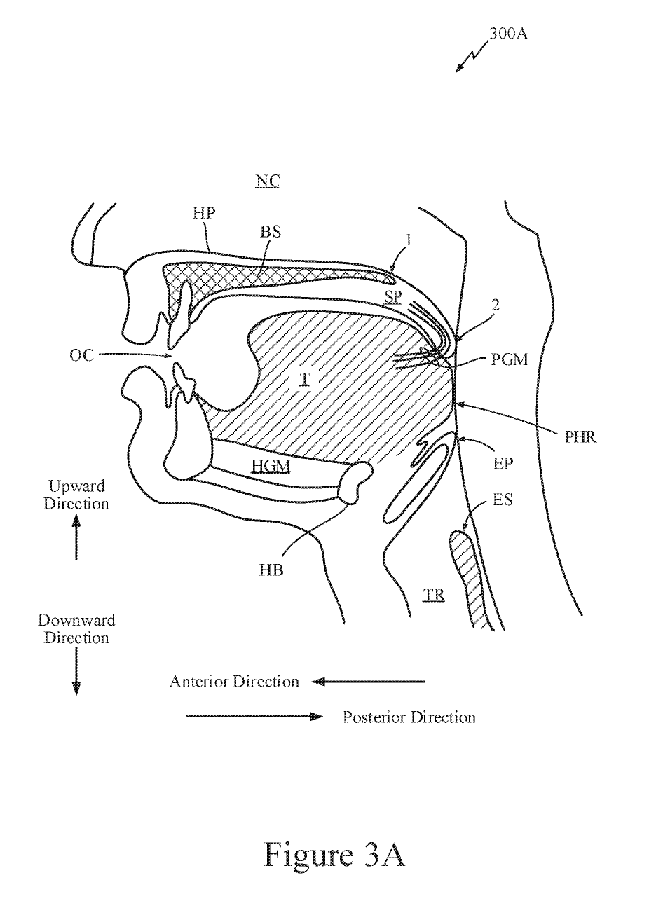

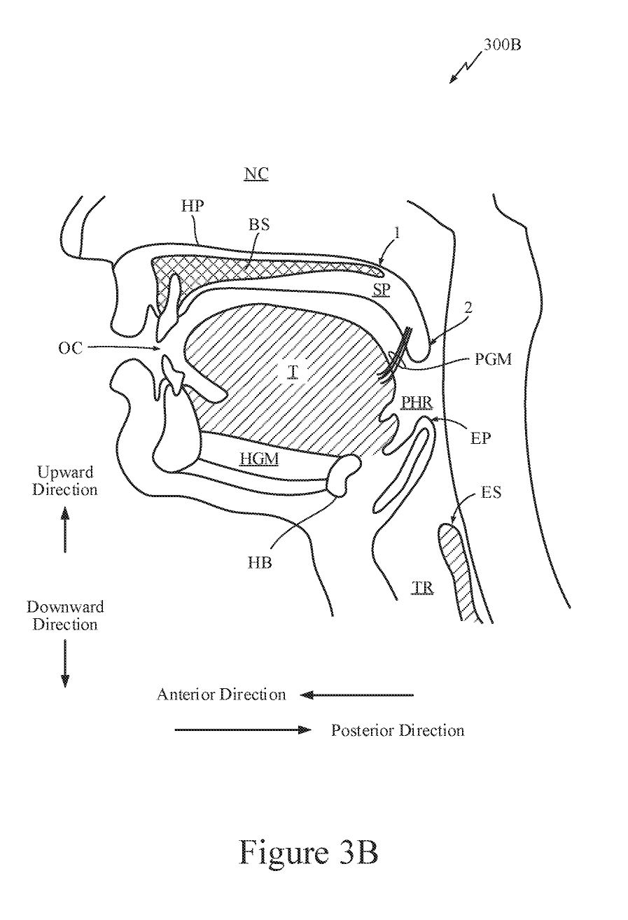

For example, FIG. 3A shows a side view 300A of a patient depicting the collapse of the patient's tongue T and soft palate SP in a posterior direction toward the back of the pharynx (PHR) during disturbed breathing. As depicted in FIG. 3A, the patient's upper airway is obstructed by the tongue T prolapsing onto the back wall of the pharynx PHR and/or by the soft palate SP collapsing onto the back wall of the pharynx PHR.

In contrast, FIG. 3B shows a side view 300B of the patient depicting the patient's upper airway response to electrical stimulation provided in accordance with aspects of the present disclosure. In some implementations, electrical stimulation provided by one or more contacts 210 of the device 200 may cause the Palatoglossus muscle PGM to stiffen and shorten, which in turn may pull the patient's soft palate SP and/or palatal arches in a downward direction, thereby preventing the soft palate SP from collapsing onto the back wall of the pharynx PHR. In addition, stiffening and/or shortening the Palatoglossus muscle PGM may also cause the patient's tongue T to contract and/or cinch downward in a manner that prevents collapse of the tongue T towards the back of the pharynx PHR without substantially moving the tongue T forward in the anterior direction.

In other implementations, electrical stimulation provided by one or more contacts 210 of the device 200 may cause the Styloglossus muscle SGM to stiffen and shorten, which in turn may cause the patient's tongue T to contract and/or cinch downward in a manner that prevents collapse of the tongue T toward the back of the pharynx PHR without substantially moving the tongue T forward in the anterior direction.

The control circuit 220 may be any suitable circuit or device (e.g., a processor) that causes electrical stimulation energy to be provided to areas proximate to the lateral edges and base of the patient's tongue T via the contacts 210(1)-210(2). More specifically, the control circuit 220 may generate one or more voltage waveforms that, when provided as signals and/or drive signals to the first and second contacts 210(1)-210(2), primarily induces a current across (e.g., in a substantially lateral direction) one or more portions of the patient's upper airway (e.g., across a lingual portion of the patient's tongue T) in a manner that causes the patient's Palatoglossus muscle PGM and/or Styloglossus muscle SGM to shorten. As used herein, inducing a current across one or more portions of the patient's upper airway refers to a direction between left and right sides of the patient's oral cavity. The waveforms provided by control circuit 220 may include continuous voltage waveforms, a series of pulses, or a combination of both. The control circuit 220 may be formed using digital components, analog components, or a combination of analog and digital components.

The control circuit 220 may vary or modify the waveform in a manner that induces a reversible current across one or more portions of the patient's upper airway (e.g., across a portion of the patient's tongue T). Applicant has discovered that inducing a reversible current across one or more portions of the patient's upper airway may decrease the likelihood of patient discomfort (e.g., as compared with providing a constant current or current in a single direction). More specifically, Applicant notes that when a current is induced in the lingual tissues of the patient, the lingual tissues may experience ion or carrier depletion, which in turn may require greater voltage differentials and/or greater current magnitudes to maintain a desired level of electrical stimulation of the Palatoglossus muscle PGM and/or the Styloglossus muscle SGM. However, inducing greater voltage and/or current magnitudes to offset increasing levels of ion or carrier depletion may create patient discomfort. Thus, to prevent ion or carrier depletion of the patient's tissues, the control circuit 220 may limit the duration of pulses that induce the current 201 across the oral cavity tissues and/or may from time to time reverse the direction (e.g., polarity) of the current 201 induced across the patient's tissues.

In some aspects, the control circuit 220 may generate and/or dynamically adjust the waveform and/or drive waveform provided to the first and second contacts 210(1)-210(2) (and/or to a number of additional contacts, not shown for simplicity) in response to one or more input signals indicative of the patient's respiratory behavior and/or inputs from other characteristics and sensing methods. The input signals may be provided by one or more of the sensors 240(1)-240(2) integrated within respective contacts 210(1)-210(2).

In other aspects, sensors other than the sensors 240(1)-240(2) integrated within respective contacts 210(1)-210(2) may be used to generate the input signals. For example, FIGS. 2C-2D show another removable intraoral device 270 in accordance with aspects of the present disclosure. The device 270 may include all the elements of the device 200 of FIGS. 2A-2B, plus additional sensors 240(3)-240(4). For the example of FIGS. 2C-2D, the sensor 240(3) may be an oxygen saturation (O₂ sat) sensor that provides a signal indicative of the patient's oxygen saturation level, and the sensor 240(4) may be a vibration sensor that provides a signal indicative of the patient's respiratory activity (as measured by vibrations detected within the patient's oral cavity). In other implementations, the sensors 240(3)-240(4) may be other types of sensors including, for example, sensors that measure air composition (especially O₂ and CO₂), heart rate, respiration, temperature, head position, snoring, pH levels, and others.

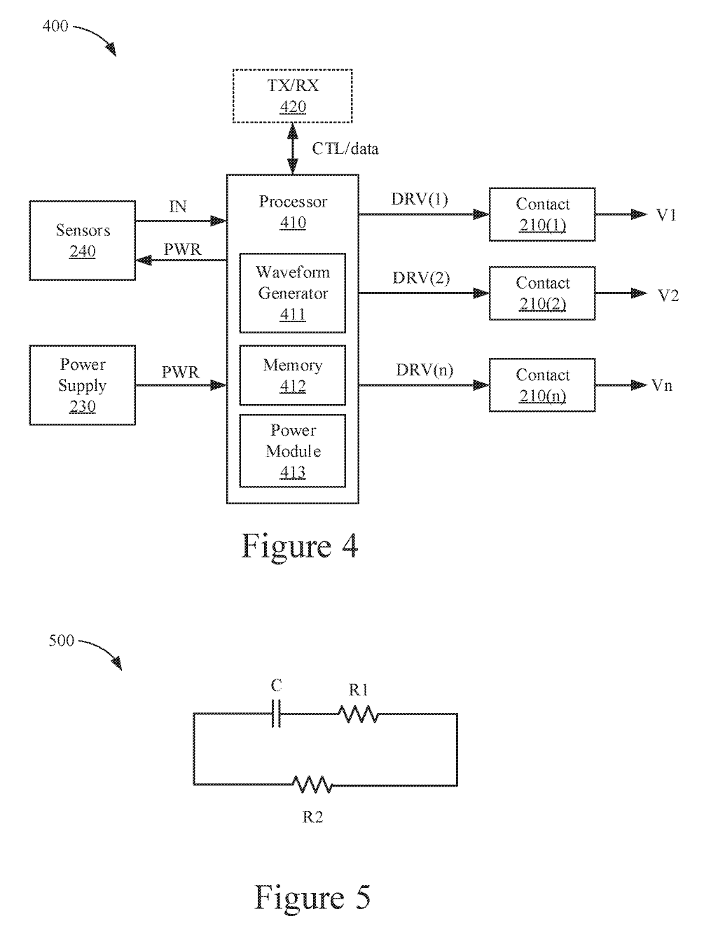

FIG. 4 shows a block diagram of the electrical components of a device 400 that may be one implementation of the device 200 of FIGS. 2A-2B. The device 400 is shown to include a processor 410, a plurality of contacts 210(1)-210(n), power supply 230, sensors 240, and an optional transceiver 420. The processor 410, which may be one implementation of the control circuit 220 of FIGS. 2A-2B, includes a waveform generator 411, a memory 412, and a power module 413. The power supply 230, which as mentioned above may be any suitable power supply (e.g., a battery), provides power (PWR) to the processor 410. In some implementations, the processor 410 may use the power module 413 to selectively provide power to the sensors 240, for example, only during periods of time that the sensors 240 are to be active (e.g., only when it is desired to receive input signals from sensors 240). Selectively providing power to the sensors 240 may not only reduce power consumption (thereby prolonging the battery life of the power supply 230) but may also minimize electrical signals transmitted along the wires 221 to the processor 410. In other implementations, the power supply 230 may provide power directly to the sensors 240.

The sensors 240, which may include the sensors 240(1)-240(2) of FIGS. 2A-2B and/or the sensors 240(3)-240(4) of FIGS. 2C-2D, may provide input signals to the processor 410. The input signals may be indicative of the respiratory behavior or other functions of the patient and may be used to detect the presence and/or absence of disturbed breathing, for example, as described above with respect to FIGS. 2A-2D. In some aspects, the input signals may be indicative of snoring in the patient.

The processor 410 may receive one or more input signals from the sensors 240, or sensors located elsewhere, and in response thereto may provide control signals and/or drive signals (DRV) to a number of the contacts 210(1)-210(n). In some implementations, the control signals and/or drive signals (e.g., voltage and/or current waveforms) generated by the waveform generator 411 may cause one or more of the contacts 210(1)-210(n) to electrically stimulate one or more portions of the patient's oral cavity OC in a manner that shortens the patient's Palatoglossus muscle PGM. Shortening the Palatoglossus muscle PGM in response to electrical stimulation provided by one or more of the contacts 210(1)-210(n) may (1) stiffen and reduce the volume of the tongue T, (2) may cause the tongue to cinch downward, and (3) may cause the Palatoglossal arch PGA to pull down (e.g., in a downward direction) towards the base of the tongue T. In this manner, the electrical stimulation provided by the one or more contacts 210(1)-210(n) may prevent the tongue T from prolapsing onto the back of the pharynx PHR and/or may prevent the soft palate SP from collapsing onto the back of the pharynx PHR and/or may prevent the tissues from vibrating.

In other implementations, the control signals and/or drive signals (e.g., voltage and/or current waveforms) generated by waveform generator 411 may cause one or more of the contacts 210(1)-210(n) to electrically stimulate one or more portions of the patient's oral cavity OC in a manner that shortens the patient's Styloglossus muscle SGM. Shortening the Styloglossus muscle SGM in response to electrical stimulation provided by one or more of the contacts 210(1)-210(n) may (1) stiffen and reduce the volume of the tongue T, (2) may cause the tongue to cinch downward, (3) may elevate the tongue toward the roof of the oral cavity (such as away from the pharynx), and (4) may retract a tip of the tongue. In this manner, the electrical stimulation provided by one or more of the contacts 210(1)-210(n) may prevent the tongue T from prolapsing onto the back of the pharynx PHR and/or may prevent the tissues from vibrating.

As mentioned above, the waveforms generated by the waveform generator 411, when provided as signals and/or drive signals to the contacts 210(1)-210(n), primarily induce a current across the patient's upper airway in a manner that causes the patient's Palatoglossus muscle PGM and/or Styloglossus muscle SGM to shorten. The waveforms generated by the waveform generator 411 may include continuous (analog) voltage waveforms, any number of pulses that may vary in shape and duration as a pulse train, or the pulses may be combined to simulate an analog waveform or a combination of both, and may be dynamically modified by the waveform generator 411. In other implementations, the waveforms generated by the waveform generator 411 may be digital pulses.

The optional transceiver 420 may be used to transmit control information (CTL) and/or data, and/or receive control information and/or data from an external device via a suitable wired or wireless connection. The external device (not shown for simplicity) may be any suitable display device, storage device, distribution system, transmission system, and the like. For one example, the external device may be a display (e.g., to display the patient's respiratory behavior or patterns, to alert an observer to periods of electrical stimulation, to indicate an alarm if breathing stops, and so on).

For another example, the external device may be a storage device that stores any data produced by the device 200, perhaps including the patient's respiratory behavior, the electrical stimulation provided by the device 200, the waveforms provided by waveform generator 411, and/or relationships between two or more of the above. In some implementations, the external device may store data for a plurality of patients indicating, for example, a relationship between the application of electrical stimulation to the patient and the patient's respiratory response to such electrical stimulation, and may include other information. Such relationship data for large numbers of patients may be aggregated, and thereafter used to identify trends or common components of OSA across various population demographics. The storage device may be a local storage device, or may be a remote storage device (e.g., accessible via one or more means and/or networks including but not limited to such as a wide area network (WAN), a wireless local area network (WLAN), a virtual private network (VPN), and/or the Internet). The data and information may be made available and/or manipulated locally and/or remotely, and may be utilized immediately and/or preserved for later utilization and/or manipulation.

The memory 412 may include a non-transitory computer-readable storage medium (e.g., one or more nonvolatile memory elements, such as EPROM, EEPROM, Flash memory, a hard drive, etc.) that may store the following software modules and/or information:
a function select module to selectively switch an active function of the contacts 210 between an electrode mode (e.g., provided by one or more of the contacts 210 and a sensor mode (e.g., provided by one or more of the sensors 240 and/or one or more of the contacts 210);
a control module to selectively provide control signals and/or drive signals to the contacts 210, for example, to induce a current across a portion of the patient's oral cavity in accordance with aspects of the present disclosure and/or to receive input signals from the sensors 240 and/or the contacts 210; and
a data collection module to record data indicative of the patient's respiratory or other behavior and/or to transmit such data to an external device.

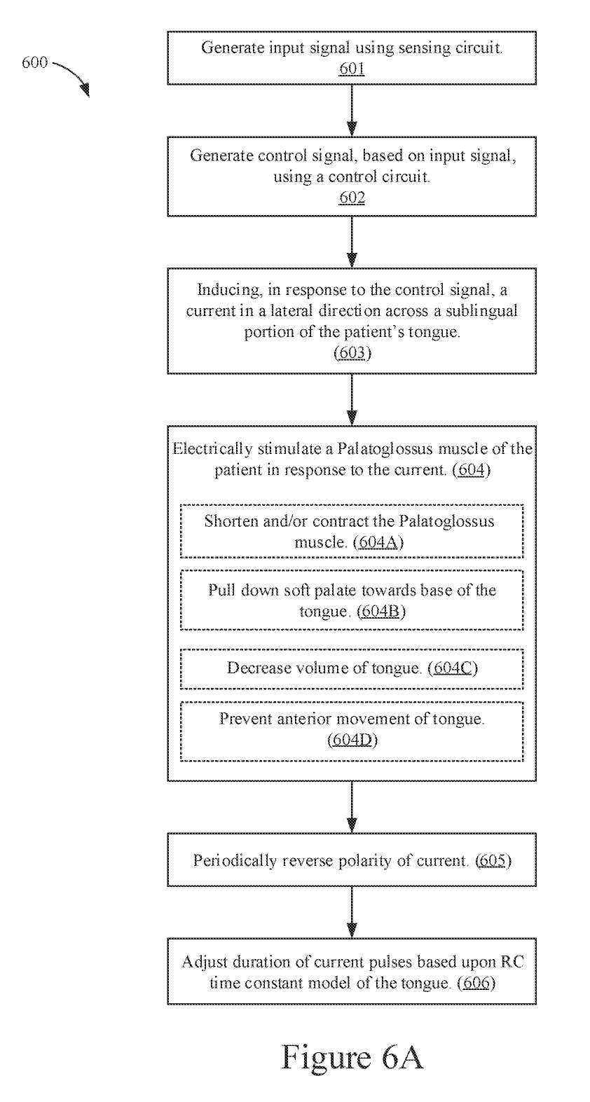

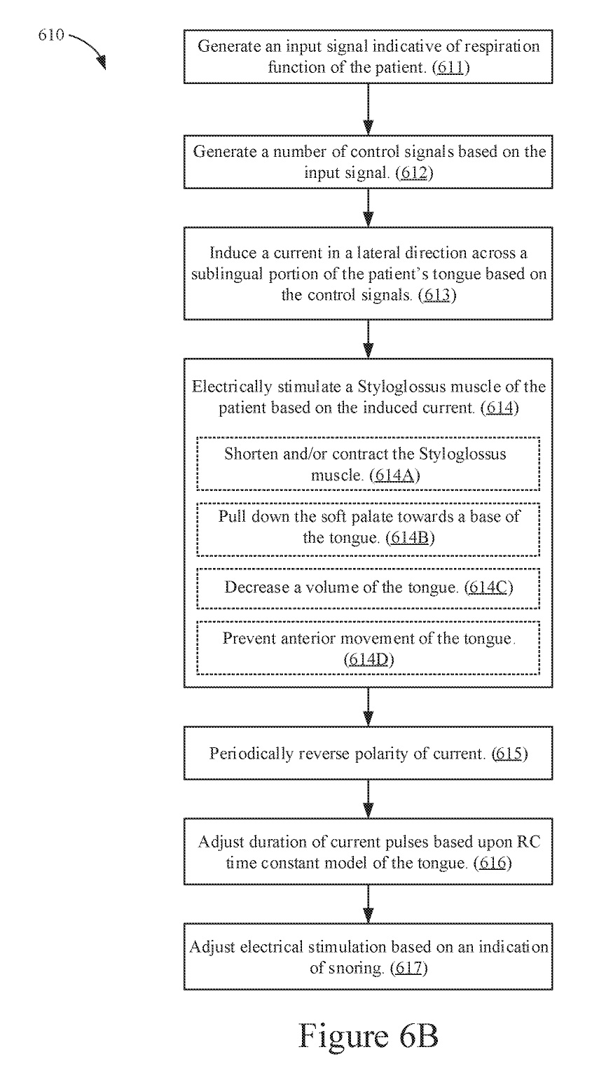

Each software module may include instructions that, when executed by the processor 410, may cause the device 400 to perform the corresponding function. Thus, the non-transitory computer-readable storage medium of the memory 412 may include instructions for performing all or a portion of the operations described below with respect to FIGS. 6A-6B. The processor 410 may be any suitable processor capable of executing scripts of instructions of one or more software programs stored in the device 400 (e.g., within memory 412). In some aspects, the memory 412 may include or be associated with a suitable volatile memory, for example, to store data corresponding to the patient's respiratory functions and/or corresponding to the electrical stimulation provided by the device 400.

Execution of the function select module may cause the processor 400 to provide one or more control signals to the contacts 210 during a first mode (such as to electrically stimulate the Palatoglossus muscle and/or the Styloglossus muscle), and may cause the processor 400 to receive an input signal indicative of a respiration function of the patient during a second mode. The input signal may be provided by the contacts 210, the sensors 240, or both. In some implementations, the processor 400 may dynamically adjust the control signals in response to the input signal, for example, to adjust the electrical stimulation based on the respiration function indicated by the input signal. In some aspects, the input signal may be indicative of snoring in the patient, and the processor 400 may commence or terminate the electrical stimulation based on whether the input signal indicates snoring in the patient.

As mentioned above, the control circuit 220 may control the duration of pulses that induce the current 201 across the patient's oral cavity, for example, to minimize carrier depletion within the patient's lingual tissues and/or may from time to time reverse the direction of the induced current 201, for example, to provide a zero sum drive waveform (e.g., to minimize or preclude electrochemical activity and/or to minimize the patient's awareness of any electrical activity related to the device 200). In some aspects, the control circuit 220 may select the pulse lengths (and/or other characteristics of the waveforms) based upon a resistive-capacitive (RC) time constant model of the patient's tongue T.

FIG. 5 shows an RC time constant model 500 of the patient's tongue T. The model 500 is shown to include a capacitor C and two resistors, R1 and R2. In one implementation, the capacitor C may be approximately 0.5 uF, the resistor R1 may be approximately 600 ohms, and the resistor R2 may be approximately 4,000 ohms. These values may result in a time constant τ=R1*C=300 μs. The resistor R2 represents minor “DC current” flow in the model, where the current stabilizes at a small but non-zero value after more than 5 time constants or when DC is applied to the contacts.

More specifically, Applicant has discovered that a typical patient's tongue T is often most receptive to a current “pulse duration” that is equal to or shorter than a time period approximately equal to τ=R1*C≈300 μs. After the time period 3τ≈1 ms expires, the patient's tongue T may exhibit an even greater increase in impedance, or perhaps experience ion depletion, which in turn requires greater voltage levels to continue inducing the current 201 across the patient's upper airway tissues. As noted above, increasing the voltage levels to continue inducing the current 201 across the patient's upper airway tissues may not only waste battery or wired power but also may cause discomfort (or even pain) to the patient. Indeed, because current regulators typically utilize their available voltage “headroom” to increase the drive voltage and maintain a constant current flow when the load impedance increases or when the effective drive voltage otherwise decreases, it is important to dynamically manage the effective drive voltage provided by the contacts 210(1)-210(2).

The effective drive voltage may decrease when there is an increased impedance, or perhaps ion depletion, in the patient's tongue, and the drive resistance may increase when one (or both) of the contacts 210(1)-210(2) loses contact with the patient's tissues, generally causing the control circuit 220 to increase its drive voltage in an attempt to maintain a prescribed current flow. Thus, in some aspects, the control circuit 220 may be configured to limit the drive voltage and/or the current to levels that are known to be safe and comfortable for the patient, even if the drive impedance becomes unusually high. In addition, the control circuit 220 may be configured to from time to time reverse the polarity or direction of the induced current 201. The reversal of the current 201 can be performed at any time. The timing of the reversal of current 201 may be selected such that there is no net transfer of charge across the patient's tissues (e.g., a zero-sum waveform).

FIG. 6A is a flow chart depicting an example operation 600 for providing electrical stimulation to a patient in accordance with aspects of the present disclosure. Although the operation 600 is discussed below with respect to the example device 200 of FIGS. 2A-2B, the operation 600 is equally applicable to other devices disclosed herein. Prior to operation, the device 200 is positioned within a sublingual portion of the patient's oral cavity, for example, so that the contacts 210(1)-210(2) are positioned on opposite lateral sides of the patient's tongue proximate to the lateral posterior regions (e.g., points) 101 at which the Palatoglossus muscle PGM and the Styloglossus muscle SGM insert into the tongue T (see also FIGS. 1A-1B). In some aspects, at least a portion of contacts 210(1)-210(2) may extend beyond a last molar location (such as in the posterior direction) of the patient's oral cavity. In other aspects, the device 200 can be positioned within an upper portion of the patient's oral cavity, for example, so that the contacts 210(1)-210(2) are positioned on opposite sides of the patient's tongue above the lateral posterior regions (e.g., points) 101 at which the Palatoglossus muscle PGM and the Styloglossus muscle SGM insert into the tongue T (see also FIGS. 1A-1B).

Once the device 200 is properly fitted within the patient's oral cavity, the device 200 accepts zero or more input signals using a number of sensing circuits provided on or otherwise associated with device 200 (601). As discussed above, the input signals may be indicative of the respiratory state or other behavior of the patient, and may be derived from or generated by any suitable sensor. The control circuit 220 generates a number of control and/or drive signals based on the input signals (602).

In response to the signals and/or drive signals, the contacts 210(1)-210(2) induce a current in a lateral direction across a portion of the patient's tongue (603). In some aspects, the current can be induced in a lateral direction across a sublingual portion of the patient's tongue. The current induced across the portion of the patient's tongue electrically stimulates the patient's Palatoglossus muscle (604). As described above, electrically stimulating the patient's Palatoglossus muscle may shorten the Palatoglossus muscle (604A), may pull down the patient's soft palate towards the base of the tongue (604B), may decrease the volume of the tongue (604C), and/or may prevent anterior movement of the tongue (604D).

In other implementations, the current induced across the portion of the patient's tongue may target the patient's Styloglossus muscle SGM. For example, FIG. 6B is a flow chart depicting another example operation 610 for providing electrical stimulation to a patient in accordance with aspects of the present disclosure. Although the operation 610 is discussed below with respect to the device 200 of FIGS. 2A-2B, the operation 610 is equally applicable to other devices disclosed herein. Prior to operation, the device 200 is positioned within a suitable portion of the patient's oral cavity, for example, so that the contacts 210(1)-210(2) are positioned on opposite sides of the patient's tongue proximate to the lateral posterior regions (e.g., points) 101 at which the Palatoglossus muscle PGM and the Styloglossus muscle SGM insert into the tongue T (see also FIGS. 1A-1B). In some aspects, at least a portion of contacts 210(1)-210(2) may extend beyond a last molar location (such as in the posterior direction) of the patient's oral cavity. In other aspects, the device 200 can be positioned within an upper portion of the patient's oral cavity, for example, so that the contacts 210(1)-210(2) are positioned on opposite sides of the patient's tongue above the lateral posterior regions (e.g., points) 101 at which the Palatoglossus muscle PGM and the Styloglossus muscle SGM insert into the tongue T (see also FIGS. 1A-1B).

Once the device 200 is properly fitted within the patient's oral cavity, the device 200 may generate one or more input signals indicative of a respiration function of the patient (611). In some aspects, the one or more input signals may be provided by sensors (such as sensors 240(3) of FIG. 2C). In other aspects, the one or more input signals may be provided by the contacts 210(1)-210(2) of device 200 or device 270 (611). As discussed above, the input signals may be indicative of any suitable respiratory state or other behavior of the patient (such as snoring), and may be derived from or generated by any suitable sensor or contact.

The control circuit 220 generates a number of control signals based on the sensing signals (612). The control signals may be generated by the waveform generator 411, and may include continuous (analog) voltage waveforms, any number of pulses that may vary in shape and duration as a pulse train, or the pulses may be combined to simulate an analog waveform or a combination of both. In some aspects, the control signals may be dynamically modified by the waveform generator 411. In other aspects, the waveforms generated by the waveform generator 411 may be digital pulses.

In response to the control signals, the contacts 210(1)-210(2) induce a current in a lateral direction across a portion of the patient's tongue (613). In some aspects, the current can be induced in a lateral direction across a sublingual portion of the patient's tongue. The current induced across the portion of the patient's tongue electrically stimulates the patient's Styloglossus muscle (614). As described above, electrically stimulating the patient's Styloglossus muscle SGM may shorten the Styloglossus muscle SGM (604A), may elevate the base of the tongue away from the pharynx, may retract the tongue from the tip (604B), may decrease the volume of the tongue (604C), and/or may prevent anterior movement of the tongue (604D).

In some implementations, the induced current may be a reversible current. In some aspects, the reversible current may be a zero-sum waveform, and the control circuit 220 may, from time to time, reverse a polarity of the reversible current (615), and/or may adjust the duration and/or amplitude of voltage and/or current pulses and/or waveforms based on the RC time constant model of the patient's tongue (616).

In addition, or as an alternative, the processor 400 may adjust the electrical stimulation based on an indication of snoring in the patient (617). In some implementations, the processor 400 may provide one or more control signals to the contacts 210 during a first mode (such as to electrically stimulate the Palatoglossus muscle and/or the Styloglossus muscle), and may receive an input signal indicative of a respiration function of the patient during a second mode. The input signal may be provided by the contacts 210, the sensors 240, or both. The processor 400 may dynamically adjust the control signals in response to the input signal, for example, to adjust the electrical stimulation based on the respiration function indicated by the input signal. In some aspects, the input signal may be indicative of snoring in the patient, and the processor 400 may commence or terminate the electrical stimulation based on whether the input signal indicates snoring in the patient.

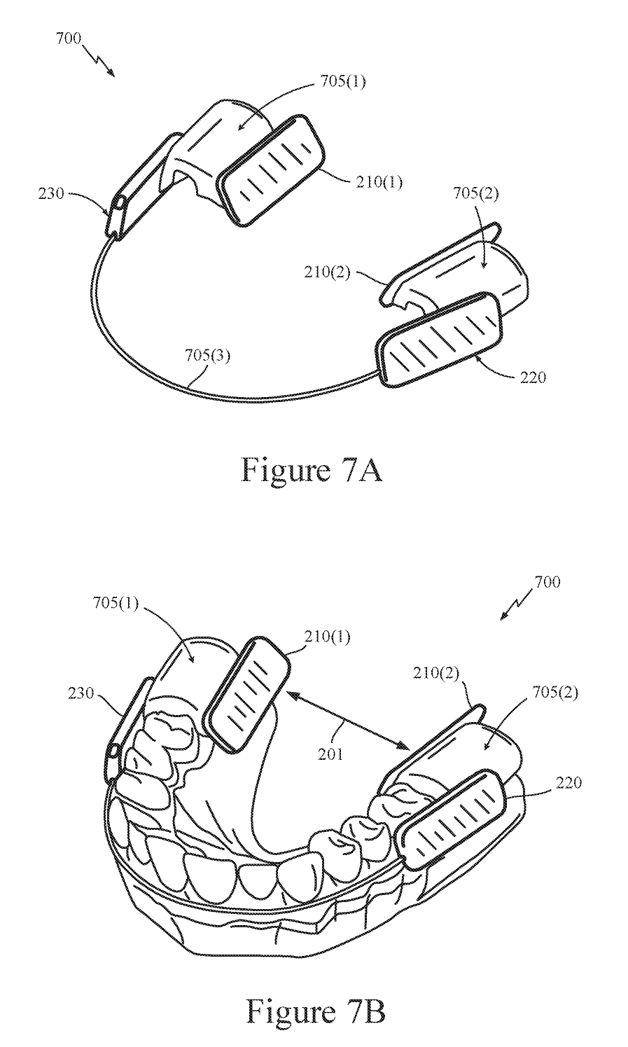

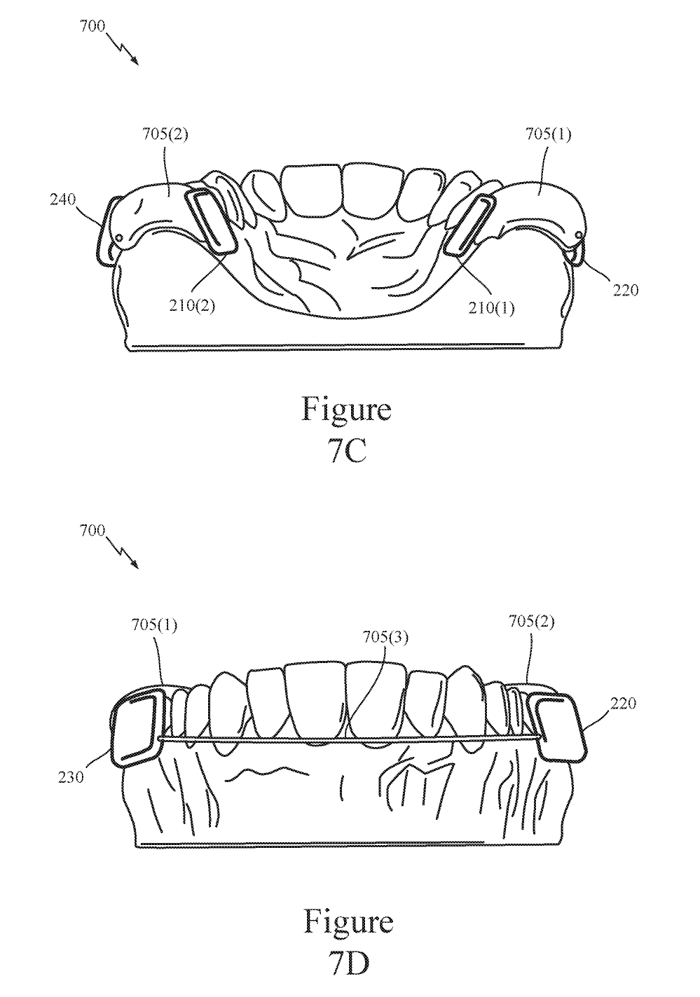

FIGS. 7A-7D show another removable intraoral device 700 in accordance with aspects of the present disclosure. The device 700 may be used to treat OSA (and/or other types of disordered breathing, discussed in more detail below with respect to FIGS. 8A-8B, 9A-9F, and 10A-10F) by providing electrical stimulation to a patient's oral cavity tissues (including, for example, the sublingual tissues) in a manner that causes the Palatoglossus muscle and/or the Styloglossus muscle SGM to shorten. The device 700 is shown to include an appliance 705 (which includes portions 705(1)-705(3), as shown in FIGS. 7C-7D) upon which contacts 210(1)-210(2), the control circuit 220, and the power supply 230 may be mounted (or otherwise attached to) so as to form a unitary and removable device that may fit entirely within a patient's oral cavity OC (see also FIGS. 1A-1B). The device 700, which may operate in a similar manner as the device 200 of FIGS. 2A-2B, includes the appliance 705 instead of the appliance 205 of FIGS. 2A-2B. Specifically, the appliance 705 includes two anchor portions 705(1)-705(2) and a support wire 705(3). The anchor portions 705(1)-705(2) may be fitted over opposite or approximately opposite molars of the patient, with the support wire 705(3) connected between anchor portions 705(1)-705(2) and extending along the patient's gum line. In other implementations, the appliance 705 may be attached, inserted, or otherwise positioned within the patient's oral cavity in any technically feasible manner.

More specifically, for the example implementations disclosed herein, the first contact 210(1) may be attached to or otherwise associated with the first anchor portion 705(1), and the second contact 210(2) may be attached to or otherwise associated with the second anchor portion 705(2). In other implementations, one or both of the anchor portions 705(1)-705(2) may be omitted (e.g., the appliance 705 may be a “floating” system in which the contacts 210(1)-210(2) are positioned within the patient's oral cavity without anchors that fit over the patient's teeth). The control circuit 220 may be attached to support wire 705(3) and/or the second anchor portion 705(2), and the power supply 230 may be attached to the support wire 705(3) and/or the first anchor portion 705(1) and/or the second anchor portion 705(2). The wires 221 (not shown in FIGS. 7A-7D for simplicity) may be attached to or provided within the support wire 705(3).

In the foregoing specification, various aspects of the present disclosure have been described with reference to specific example implementations. It will, however, be evident that various modifications and changes may be made thereto without departing from the broader scope of the disclosure as set forth in the appended claims. The specification and drawings are, accordingly, to be regarded in an illustrative sense rather than a restrictive sense.

What is claimed is:

1. A device comprising:
an appliance configured to fit within an oral cavity of a patient; and
a number of contacts, coupled to the appliance and adapted to be positioned on opposite lateral sides of the patient's tongue, and configured to electrically stimulate a Styloglossus muscle of the patient without targeting the patient's Hypoglossal nerve, wherein a portion of at least one of the contacts is adapted to be positioned posterior to a last molar location of the patient.

2. The device of claim 1, wherein the number of contacts are configured to induce a current in a lateral direction across the tongue the current by providing a voltage differential across the tongue.

3. The device of claim 2, wherein the induced current comprises one or more reversible currents configured to flow in one or more substantially lateral directions across the tongue.

4. The device of claim 3, wherein a pulse length of at least one of the reversible currents is configured according to a resistive-capacitive (RC) time constant model associated with the tongue.

5. The device of claim 1, wherein at least a portion of one or more of the contacts is adapted to be angularly oriented with respect to a floor of the patient's mouth.

6. The device of claim 1, wherein one or more of the contacts are adapted to be in contact with one or more lateral points at which the Styloglossus muscle inserts into the tongue.

7. The device of claim 1, further comprising:
a control circuit, coupled to the appliance, to generate a signal, wherein the contacts are responsive to the signal.

8. The device of claim 7, further comprising:
a power supply, mounted on the appliance, to provide power to the control circuit, wherein the appliance, the contacts, the control circuit, and the power supply comprise a unitary device adapted to fit entirely within the patient's oral cavity.

9. The device of claim 1, wherein the electrical stimulation is configured to shorten the Styloglossus muscle without targeting the patient's Hypoglossal nerve.

10. The device of claim 9, wherein the electrical stimulation is further configured to stiffen the tongue, to elevate a posterior portion of the tongue towards a roof of the patient's oral cavity, and to retract a tip of the tongue.

11. The device of claim 1, wherein the electrical stimulation is further configured to decrease a volume of the tongue without moving the tongue in an anterior direction.

12. The device of claim 1, wherein one or more of the contacts comprises an electromyogram (EMG) sensor configured to detect electrical activity of muscles within or connected to the patient's tongue.

13. The device of claim 1, wherein the electrical stimulation is configured to avoid targeting a genioglossus muscle of the patient.

14. The device of claim 1, wherein the device is removable.

15. The device of claim 1, wherein at least one of the contacts is further configured to sense a respiration function of the patient.

16. The device of claim 1, further comprising a control circuit configured to:
provide one or more first signals to the at least one of the contacts during a first mode, the one or more first signals configured to provide the electrical stimulation; and
receive a second signal from the at least one of the contacts during a second mode, the second signal indicative of the patient's respiration function.

17. The device of claim 16, wherein the second signal is indicative of snoring in the patient, and the control circuit is configured to commence the electrical stimulation based on the indication of snoring.

18. The device of claim 16, wherein the control circuit is further configured to dynamically adjust the one or more first signals in response to the second signal.

19. The device of claim 1, wherein the number of contacts are further configured to electrically stimulate a Palatoglossus muscle of the patient while electrically stimulating the Styloglossus muscle.

20. The device of claim 19, wherein the device is configured to electrically stimulate the Palatoglossus muscle and the Styloglossus muscle at staggered times.
